(19) United States
(12) Patent Application Publication
(10) Pub. No.: US 2026/0283803 A1
(43) Pub. Date: Sep. 24, 2026

(54) SYSTEMS AND METHODS FOR ENGINEERING MICRO AND NANOSCALE TEXTURE ON IMPLANTS

(71) Applicant: Warsaw Orthopedic, Inc., Warsaw, IN (US)

(72) Inventors: Michelle B. Gallagher, Fuquay-Varina, NC (US); Olumide O. Aruwajoye, Missouri City, TX (US); Larry T. McBride, Germantown, TN (US); Yana Rawinski, Shorewood, WI (US); James T. Sugar, Milwaukee, WI (US); Thomas G. Zipp, Green Bay, WI (US); William F. Lundin, Elm Grove, WI (US); Phillip S.E.R. Solomon, Milwaukee, WI (US); Chris Italiaie, Playa Del Rey, CA (US)

(21) Appl. No.: 19/475,072

(22) PCT Filed: Apr. 18, 2024
(86) PCT No.: PCT/IB2024/053801

§ 371 Date: Oct. 14, 2025

Related U.S. Application Data
(60) Provisional application No. 63/497,038, filed on Apr. 19, 2023.

(51) Int. Cl. A61F 2/30; A61B 17/70

(52) U.S. Cl. CPC A61F 2/3094; A61B 17/7001; A61F 2002/30838; A61F 2002/3084; A61F 2002/3093

(57) ABSTRACT

System and methods (300) for adding surface roughness to an implant (100, 400) with an anodized surface finish. The methods comprising: masking first and second portions (402, 404) of the implant to obtain a maskant-implant assembly (1600) with a tight seal formed around the second portion of the implant; roughening a surface of a third portion of implant using a maskant-implant-holder assembly (2700); and removing a maskant and a holder (2000) from the implant, wherein the anodized surface finish remains on the first and second portions of the implant and has been removed from the third portion (406, 416) of the implant.

### BACKGROUND

This application claims the benefit of U.S. Provisional Patent Application Ser. No. 63/497,038, filed 19 Apr. 2023, the entire content of which is incorporated herein by reference.

Implants are often comprised of metal and metal alloys (e.g., titanium (Ti) and titanium alloys). For some implants to function successfully, sufficient osseointegration is required so that a bond between the implant and the bone is formed and retained.

### SUMMARY

The present disclosure concerns implementing systems and methods for adding surface roughness to an implant (e.g., a screw) with an anodized surface finish. The methods comprise, for example: masking first and second portions (e.g., a head and neck) of the implant to obtain a maskant-implant assembly; loading the maskant-implant assembly into a holder to obtain a maskant-implant-holder assembly with a tight seal formed around the second portion of the implant; roughening a surface of a third portion (e.g., a shank and/or threads) of implant using the maskant-implant-holder assembly; and/or removing a maskant (e.g., a cap mask) and the holder from the implant, wherein the anodized surface finish remains on the first and second portions of the implant and has been removed from the third portion of the implant.

The methods may also comprise masking an internal feature of the implant using one or more an insert masks. The internal feature may be masked by: stretching the insert mask; pulling the insert mask through an aperture formed in the implant; and releasing the insert mask so that the insert mask at least partially unstretches and seals the aperture.

The masking step may involve loading a cap mask on the first and second portions of the implant. The cap mask may be loaded on the first and second portions of the implant by: causing the cap mask to at least partially deform from an initial state so that the first portion of the implant is able to slide through an insert opening and into an internal space of the cap mask; and allowing the cap mask to at least partially return to the initial state whereby the first portion of the implant resides in the internal space of the cap mask and the cap mask encompasses and tightly fits around the second portion of the implant.

The maskant-implant assembly may be loaded into the holder by: applying a first pushing force on the maskant-implant assembly to cause a cap mask to slide through an opening of an aperture formed in the holder; discontinuing application of the first pushing force when at least one flange of the cap mask slides past a flange formed at the opening of the aperture formed in the holder; using a tool to apply a second pushing force to the maskant-implant assembly to cause the cap mask to further slide into the aperture formed in the holder; and discontinuing application of the second pushing force when the cap mask is fully loaded in the aperture formed in the holder. The cap mask may entirely reside in the aperture formed in the holder when the maskant-implant assembly is loaded into the holder. The methods may further comprise using one or more mechanisms to hold the cap mask in the holder during the surface roughening process(es). These mechanisms can include, for example, (1) suction created at least partially by the flange(s) of the cap mask that facilitates retention of the cap mask in the aperture formed in the holder, and/or (2) the flange formed at the opening of the aperture formed in the holder that provides a primary hold on the cap mask. The purpose of the two listed mechanisms is to ensure that the cap mask and holder do not move with respect to each other, which provides a consistent external pressure on the masking through processing.

The roughening may create microscale features (e.g., micron scale valleys) on a surface of the second portion (e.g., the neck) and/or third portion (e.g., the shaft) of the implant and/or nanoscale features within or around the microscale features (e.g., nanoscale striations within the micron scale valleys). The geometric dimensions of the first and/or second portions of the implant may be the same or different before and after the roughening of the surface of the third portion.

The disclosure also concerns a system for use with an implant during a surface roughening process. The system comprises: a cap mask configured to maintain geometric dimensions and an anodized surface finish of first and second portions of the implant during the surface roughening process in which surface roughness is added to a third portion of the implant; a holder with an aperture sized and shaped to receive the cap mask in a manner that facilitates creation of a seal around the second portion of the implant; and a structure provided with the cap mask to facilitate retention of the cap mask in the holder when the cap mask is fully inserted into the aperture of the holder.

The surface roughening process may comprise at least one of grit blasting and/or chemical etching. The implant may comprise a screw. The first portion may comprise a head of the screw. The second portion may comprise a neck of the screw. The third portion may comprise a shaft and threads of the screw.

The system may also comprise an insert mask configured to mask and seal an internal feature of the implant during the surface roughening process. The insert mask may be formed of a resilient material that deforms when a pulling force is applied thereto and undeforms when the pulling force is no longer being applied thereto.

The cap mask and holder can be configured to allow removal of the anodized surface finish from the third portion of the implant during the surface roughening process. The cap mask may be further configured to: at least partially deform from an initial state whereby an insert opening of the cap mask is enlarged to a size and shape for slidingly receiving the first portion of the implant; and at least partially return to the initial state whereby the insert opening of the cap mask decreases in size to cause the cap mask to encompass and tightly fit around the second portion of the implant.

The holder comprises: an aperture sized and shaped to receive the cap mask; and at least one flange that is formed at an opening of the aperture and configured to. The flange(s) of the holder may be configured to facilitate compression of the cap mask when inserted into the holder, facilitate retention of the cap mask in the aperture formed in the holder, prevent the cap mask from moving with respect to the holder for ensuring consistent mask compression, and/or provide an indication of when a suction structure of the cap mask has been inserted into the aperture of the holder. The suction structure of the cap mask may be configured to create suction to facilitate retention of the cap mask in the aperture formed in the holder.

The document further concerns an implant. The implant comprises, for example: a first portion with an anodized surface finish; a second portion that is connected to or integral with the first portion and has the anodized surface finish; and a third portion that is connected to or integral with the second portion, absent of the anodized surface finish, and has a surface roughness comprising microscale features and nanoscale features within or around the microscale features. The geometric dimensions of the first and second portions of the implant can be generally the same before, during and after addition of the surface roughness to the third portion of the implant.

### BRIEF DESCRIPTION OF DRAWINGS

The following drawings are illustrative of particular embodiments of the present disclosure and therefore do not limit the scope of the present disclosure. The drawings are not to scale and are intended for use in conjunction with the explanations in the following detailed description.

FIG. 1 provides an illustration of an implant.

FIG. 2 provides illustrations of one possible type of surface roughness applied to a surface of the implant shown in FIG. 1.

FIG. 3 provides a flow diagram of an illustrative method for adding surface roughness to an implant.

FIG. 4 provides an illustration of an implant with an anodized finish.

FIGS. 5-12 provide illustrations showing a process for masking internal features of an implant using an insert mask.

FIG. 13 provides a side view of a cap mask.

FIG. 14 provides a cross-sectional view of the cap mask shown in FIG. 13.

FIG. 15 provides an illustration that is useful for understanding how a cap-implant assembly is produced.

FIG. 16 provides a side view of a cap-implant assembly.

FIG. 17 provides a cross-sectional view of the cap-implant assembly shown in FIG. 16. The internal feature and insert mask are not shown in FIG. 17 for ease of illustration.

FIG. 18 provides illustrations showing a cap in a fully loaded position on an implant and a cap that is not in the fully loaded position on the implant.

FIG. 19 provides an illustration showing a tool for facilitating the loading of cap-implant assembly into a holder.

FIG. 20 provides an illustration of a process for loading of cap-implant assembly into a holder using the tool of FIG. 19.

FIG. 21 provides a cross-sectional view of the cap-implant assembly and holder shown in FIG. 19.

FIG. 22 provides a cross-sectional view of the cap-implant assembly at least partially loaded into the holder.

FIG. 23 provides a cross-sectional view of the tool shown in FIG. 19 with the partially loaded cap-implant assembly coupled thereto.

FIG. 24 provides a cross-sectional view of the tool shown in FIG. 19 with the cap-implant assembly fully loaded into the holder.

FIG. 25 provides a side view of a cap-implant assembly loaded into the holder.

FIG. 26 provides a cross-sectional view of the cap-implant-holder assembly shown in FIG. 25.

FIG. 27 provides a perspective view of the cap-implant-holder assembly.

FIG. 28 provides illustrations showing a cap-implant assembly fully loaded in the holder and a cap-implant assembly that is not fully loaded in the holder.

FIG. 29 provides a perspective view of a tray.

FIG. 30 provides illustrations of the cap-implant-holder assembly being inserted into the tray of FIG. 29.

FIG. 31 provides an illustration of the implant with surface roughness.

FIG. 32 provides a graph showing results from mechanical testing of the implant with and without masking during surface processing.

FIG. 33 provides a flow diagram of another illustrative method for adding surface roughness to an implant.

FIG. 34 provides an illustration of a semi-flexible mask (e.g., liquid or putty mask) provided on an implant.

FIG. 35 provides an illustration showing a holder being loaded onto a maskant-implant assembly of FIG. 34.

FIG. 36 provides an illustration showing the holder of FIG. 35 loaded onto the maskant-implant assembly of FIG. 34.

FIG. 37 provides a cross-sectional view of the maskant-implant-holder assembly of FIG. 36.

FIG. 38 provides an illustration of a maskant for a preassembled implant with the tulip attached to the shank prior to masking.

FIG. 39 provides an illustration of a maskant applied to a preassembled implant.

FIG. 40 provides illustrations showing a process for applying a liquid maskant to an implant.

### DETAILED DESCRIPTION

The following discussion omits or only briefly describes certain conventional features related to surgical systems for treating the spine, which are apparent to those skilled in the art. It is noted that various embodiments are described in detail with reference to the drawings, in which like reference numerals represent like parts and assemblies throughout the several views. Reference to various embodiments does not limit the scope of the claims appended hereto. Additionally, any examples set forth in this specification are intended to be non-limiting and merely set forth some of the many possible embodiments for the appended claims. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

As noted above, implants are often comprised of metal and metal alloys (e.g., titanium (Ti) and titanium alloys). For some implants to function successfully, sufficient osseointegration or enhanced fixation is required so that a bond between the implant and the bone is formed and retained. The surface of the implant may be roughened to help enhance the osseointegration process. Grit blasting and/or chemical etching may be employed to impart roughness on the surface.

The present solution provides implementing systems and methods for incorporating surface roughness on a portion of the implant (e.g., the threaded shank of a spinal screw) that comes in contact with bone during and following surgery, while an anodized surface finish is maintained on other portions of the implant (e.g., the head and neck of the spinal screw). Maintaining the anodized surface finish on certain portions of the implant improves mechanical integrity and human factors feature for easy implant identification. To achieve these different regions of surface texture, a novel masking technique is employed during the grit blasting and/or chemical etching processes. The particulars of the novel masking technique will become evident as the discussion progresses. It has been shown that during in vivo testing a spinal screw produced using the novel masking technique of the present solution has an increased extraction torque at all time points when compared to a conventional anodized spinal screw. The increased extraction torque, that reflects enhanced fixation, is facilitated by the micro-and nano-textured surface of the threaded shank of the spinal screw.

Embodiments of the present disclosure generally relate to implementing systems and methods for incorporating surface roughness on a portion of the implant (e.g., the threaded shank of a spinal screw) that comes in contact with bone during and following surgery. The methods comprise: loading a cap mask on first and second portions (e.g., a head and neck) of the implant to obtain a cap-implant assembly; loading the cap-implant assembly into a holder to obtain a cap-implant-holder assembly with a seal formed around the second portion of the implant; roughening a surface of a third portion (e.g., a shank and/or threads) of implant using the cap-implant-holder assembly; and removing the cap mask and holder from the implant.

The anodized surface finish remains on the first and second portions of the implant during the surface roughening process, but is removed from the third portion of the implant during surface roughening process.

The cap mask may be loaded on the first and second portions of the implant by: causing the cap mask to at least partially deform from an initial state so that the first portion of the implant is able to slide through an insert opening and into an internal space of the cap mask; and allowing the cap mask to at least partially return to the initial state whereby the first portion of the implant resides in the internal space of the cap mask and the cap mask encompasses and tightly fits around the second portion of the implant.

The cap-implant assembly may be loaded into the holder by: applying a first pushing force on the cap-implant assembly to cause the cap mask to slide through an opening of an aperture formed in the holder; discontinuing application of the first pushing force when at least one flange of the cap mask slides past a flange formed at the opening of the aperture formed in the holder; using a tool to apply a second pushing force to the cap-implant assembly to cause the cap mask to further slide into the aperture formed in the holder; and discontinuing application of the second pushing force when the cap mask is fully loaded in the aperture formed in the holder. The cap mask may entirely reside in the aperture formed in the holder when the cap-implant assembly is loaded into the holder. The methods may further comprise using one or more mechanisms to hold the cap mask in the holder during the surface roughening process(es). These mechanisms can include, for example, (1) suction created at least partially by the flange(s) of the cap mask to facilitate retention of the cap mask in the aperture formed in the holder, and/or (2) the flange formed at the opening of the aperture formed in the holder that provides a primary hold on the cap mask. The purpose of the two listed mechanisms is to ensure that the cap mask and holder do not move with respect to each other, which provides a consistent external pressure on the masking through processing.

The roughening may create microscale features (e.g., micron scale valleys) on a surface of the second portion (e.g., the neck) and/or third portion (e.g., the shaft) of the implant and/or nanoscale features within or around the microscale features (e.g., nanoscale striations within the micron scale valleys). The geometric dimensions of the first and/or second portions of the implant may be the same or different before and after the roughening of the surface of the third portion of the implant.

The methods may also comprise masking an internal feature of the implant using an insert mask. The internal feature may be masked by: stretching or otherwise deforming the insert mask; pulling the insert mask through an aperture formed in the implant; and releasing the insert mask so that the insert mask at least partially unstretches or otherwise undeforms and seals the aperture.

The disclosure also concerns a system for use with an implant during a surface roughening process. The system comprises: a cap mask configured to maintain geometric dimensions and an anodized surface finish of first and second portions of the implant during the surface roughening process in which surface roughness is added to a third portion of the implant; a holder with (i) an aperture sized and shaped to receive the cap mask in a manner that facilitates creation of a seal around the second portion of the implant and (ii) flange(s) configured to facilitate retention of the cap mask during surface roughening process(es); and a structure provided with the cap mask to create suction when the cap mask is inserted into the aperture of the holder.

The surface roughening process may comprise at least one of grit blasting and acid etching. The implant may comprise a screw. The first portion may comprise a head of the screw. The second portion may comprise a neck of the screw. The third portion may comprise a shaft and threads of the screw.

The system may also comprise an insert mask configured to mask and seal an internal feature of the implant during the surface roughening process. The insert mask is formed of a resilient material that deforms when a pulling force is applied thereto and undeforms when the pulling force is no longer being applied thereto.

The cap mask and holder are configured to allow removal of the anodized surface finish from the third portion of the implant during the surface roughening process. The cap mask is further configured to: at least partially deform from an initial state whereby an insert opening of the cap mask is enlarged to a size and shape for slidingly receiving the first portion of the implant; and at least partially return to the initial state whereby the insert opening of the cap mask decreases in size to cause the cap mask to encompass and tightly fit around the second portion of the implant.

The holder comprises: an aperture sized and shaped to receive the cap mask; and at least one flange that is formed at an opening of the aperture and configured to facilitate compression of the cap mask when inserted into the holder and/or facilitate retention of the cap mask during surface roughening process(es). The flange(s) of the holder is(are) additionally configured to provide an indication of when a suction structure of the cap mask has been inserted into the aperture of the holder. The suction structure of the cap mask is configured to create suction to facilitate retention of the cap mask in the aperture formed in the holder.

The document further concerns an implant. The implant comprises: a first portion with an anodized surface finish; a second portion that is connected to the first portion and has the anodized surface finish; and a third portion that is connected to the second portion, absent of the anodized surface finish, and has a surface roughness comprising micron scale valleys and nanoscale striations within the micron scale valleys. The geometric dimensions of the first and second portions of the implant can be generally the same before, during and after addition of the surface roughness to the third portion of the implant.

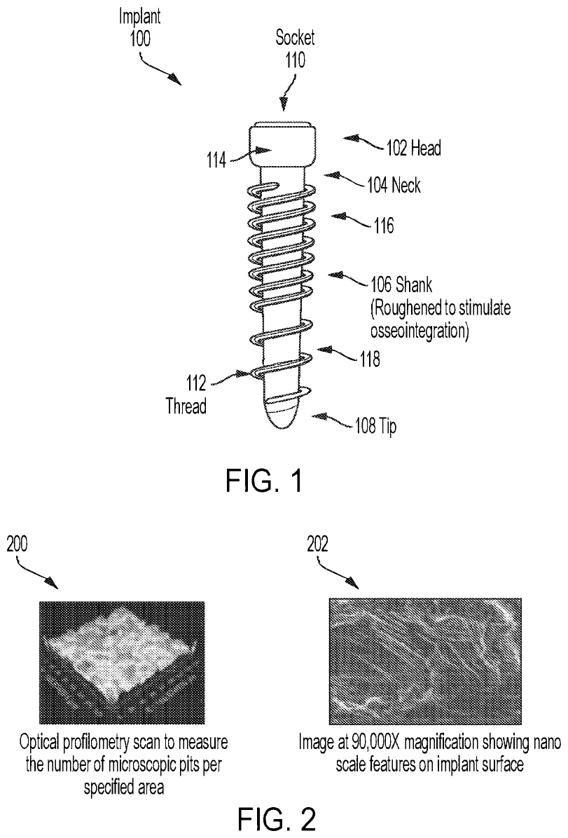

Referring now to FIG. 1, there is provided an illustration of an implant 100. Implant 100 is shown as comprising a spinal screw, a sacropelvic screw, or a pedicle screw. The spinal screw can include, but is not limited to, a ModuleX™ spinal system. ModuLeX™ spinal system shanks are designed for posterior screw fixation in spinal procedures and offer a universal connection design that allows for different head assemblies and/or tulips to be connected onto a screw shank. The present solution is not limited in this regard. Implant 100 could include other types of things that can be implanted in a person's body and/or should stimulate osteointegration. Implant 100 may, for example, be made of titanium, tantalum, cobalt, chromium, stainless steel, and/or alloys thereof. Implant 100 may also have an anodized surface finish.

Implant 100 comprises a head 102, a neck 104, and a shank 106 connecting the neck 104 to a tip 108. The head 102 includes a socket 110 that may be engaged by tool for screwing the implant 100 into bone tissue. Socket 110 can have any shape selected in accordance with a given application. For example, socket 110 can have a polygonal or hexagonal shape with flat surfaces 114 and be sized to receive a mating end of the tool. In other scenarios, the head 102 comprises a protruding boss rather than a socket, and/or has a continuous round exterior surface with no flat surfaces 114. Implant 100 may also include configurations where the head is spherical, cylindrical, conical, oval, or of other various shapes or combinations of shapes including ledges, flat surfaces or steps and may further include, for example, screws that do not have a head or may not include a traditional neck.

The fastening of the implant 100 to bone tissue is facilitated by threads 112 formed on the shank 106 and the tip 108 of the shank. The threads 112 make a plurality of turns around the shank 106. The threads 112 can have a constant or varying turn density along the length of the shank 106. For example, as shown in FIG. 1, there are a greater number of thread turns on a proximal portion 116 of the shank and a lesser number of thread turns on a distal portion 118 of the shank. This results in adjacent thread turns having different spacings on the proximal and distal portions of the shank. In other scenarios, the same number of thread turns are provided on the proximal and distal portions of the shank. The threads may alternatively be blunted, twisted and/or interrupted.

The tip 108 can have any geometry selected in accordance with a given application. For example, the tip 108 may be rounded as shown in FIG. 1 and/or more pointed, fluted, self-tapping or sharp than shown in FIG. 1. Fenestrations (not shown) and/or cannulations (not shown) may be provided that extend, for example, from the head 102 to the tip 108.

The shank 106 and threads 112 are roughened to stimulate osseointegration for creating a direct structural and functional connection between living bone and the surface of the implant. The roughness of the implant 100 is on a micron and nano level. The textured surface features are designed to mimic a bone remodeling process. The textured surface features consist of micron scale valleys shown in image 200 of FIG. 2. The micron scale valleys have dimensions correlating to osteoclastic resorption pits. Nanoscale striations or steps are formed within the micron scale valleys, as shown in image 202 of FIG. 2.

The textured surface features may be applied to the implant via grit blasting and acid etching processes. A novel masking technique is employed during the grit blasting and acid etching processes so that the anodized finish can be maintained on the neck 104 and head 102 of the implant 100. Masking of the neck 104 is performed to maintain mechanical integrity of the implant.

The anodization process does not remove material, rather it thickens the oxide layer of the metal (e.g., titanium). Maintenance of the neck's diameter in certain embodiments is critical for mechanical integrity during dynamic loading. The grit blasting and acid etching processes are a subtractive process that removes material from the neck of the implant if not masked. The present solution is designed to generate specifically engineered features at two levels which mimic the bone remodeling process. Note, the macro-scale roughness on prior interbody cage applications is not utilized for certain screw configuration as it may heavily impact the thread form.

The surface treatment of the present solution is a subtractive process that provides a microscopic-roughened surface with nano-scale features that is designed to improve fixation to the adjacent bone. The first level is the micron scale valleys with dimensions correlating to osteoclastic resorption pits (e.g., 1-1000 microns; valleys are approximately 20-100 ums in diameter and 10-30 um deep). The number of valleys ensures biomimicry at this level. The second level is nanoscale (e.g., 1-1000 nm) which consists of nano striations or steps superimposed within those micropits. The nanoscale striations/steps have a high aspect ratio, for example, with a length typically greater than 100 nm but a width less than 100 nm. They are not depressions into the material rather ridges with peaks. It is a combination of the two textures that constitute a biomimic surface which promotes an increase in bone formation.

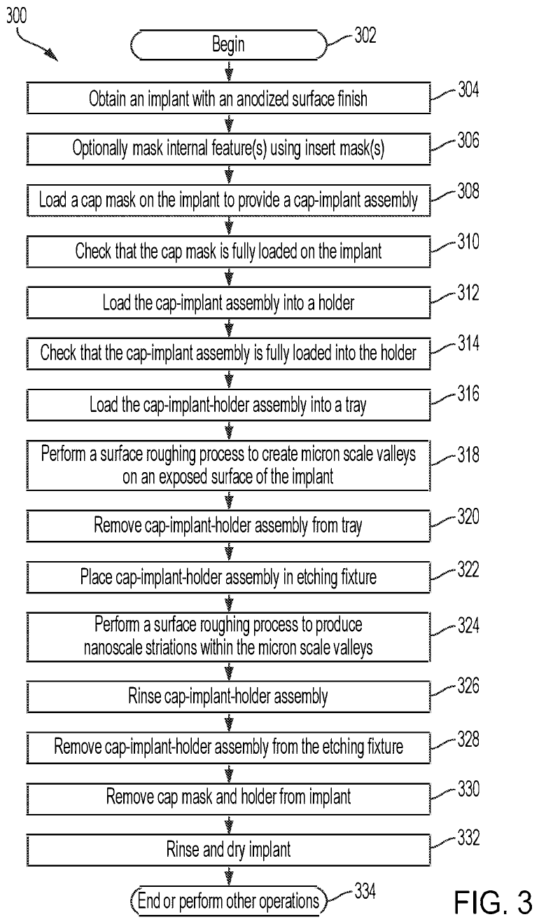

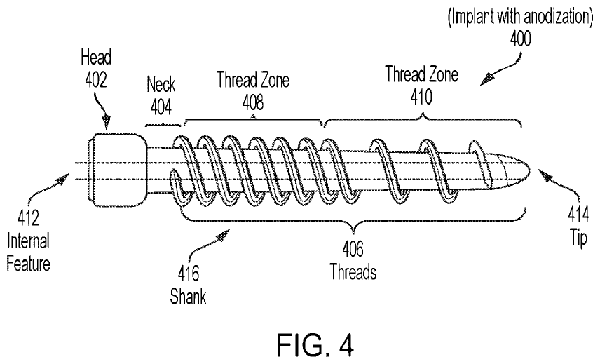

Referring now to FIG. 3, there is provided a flow diagram of an illustrative method 300 for adding surface roughness to an implant. Method 300 begins with 302 and continues with 304 where an implant is obtained with an anodized surface finish. An illustration of an implant 400 is shown in FIG. 4. Implant 400 can be the same as, similar to, and/or different than implant 100 of FIG. 1. Implant 400 comprises a head 402, a neck 404, and shank 416 with threads 406 having two thread zones 408, 410. An internal feature 412 is also provided with implant 400. The internal feature 412 comprises an elongate aperture extending from the head 402 to the tip 414. An anodized surface finish is provided on the head 402, a neck 404, shank 416 and threads 406.

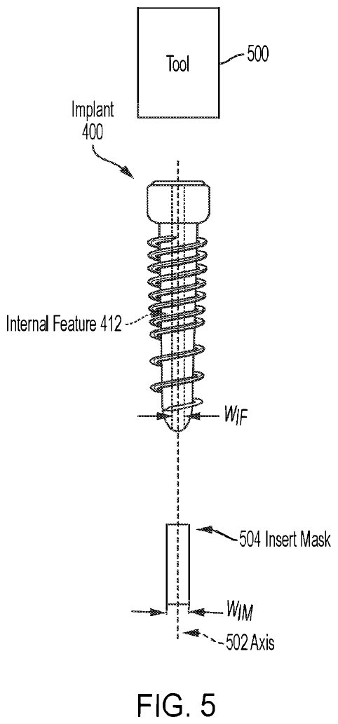

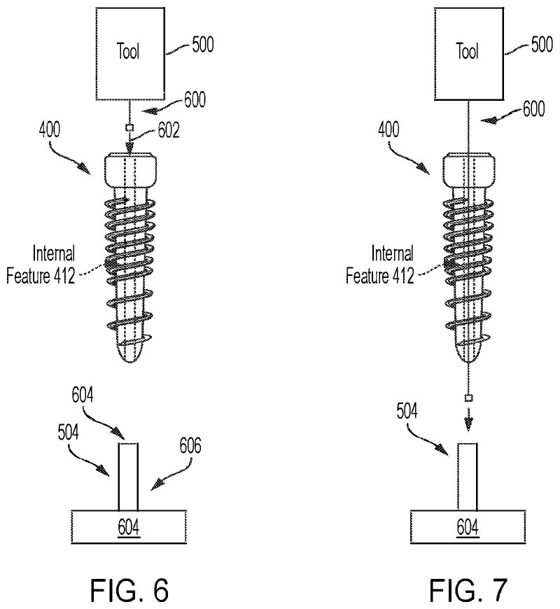

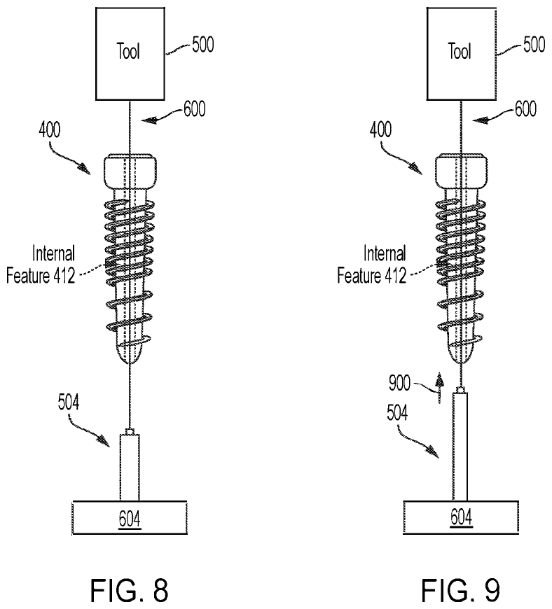

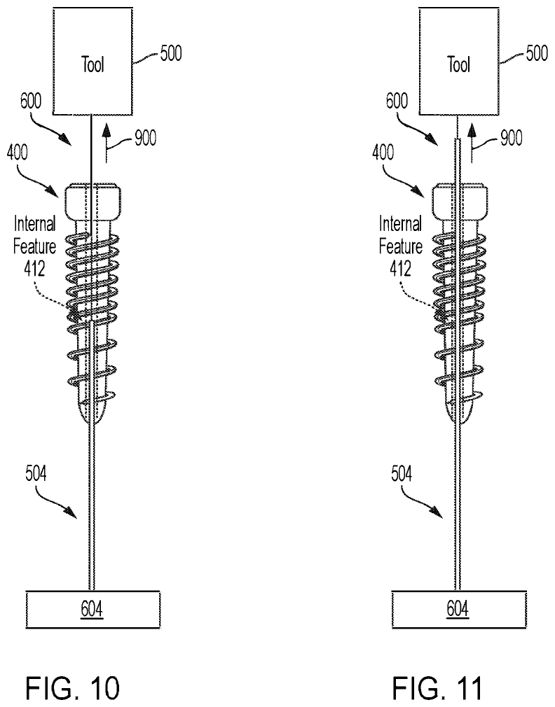

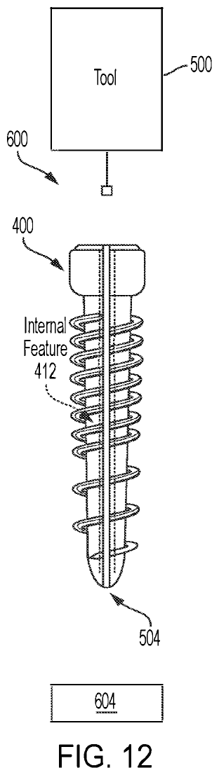

When the implant includes internal feature(s), method 300 continues with optional operations of block 306. These operations involve masking internal feature(s) using an insert mask. The particulars of the operations can be understood with reference to FIGS. 5-12 in which the internal feature 412 of implant 400 is masked using an insert mask 504. Insert mask 504 is formed of a resilient material (such as rubber) that can be stretched (or otherwise deformed) using a tool 500 and automatically return at least partially to its original unstretched (or undeformed) state when released from the tool. In the unstretched (or undeformed) state shown in FIG. 5, the insert mask 504 has a width W_{IM} that is greater than the width W_{IF} of the internal feature 412. In the fully stretched (or deformed) state shown in FIGS. 10-11, the width W_{IM} of the insert mask 504 is smaller than the width W_{IF} of the internal feature 412. This allows the tool 500 to pull the insert mask 504 in direction 900 and through the internal feature 412 as shown in FIGS. 9-11. The tool 500 can have a movable component 600 with a gripper or other connector (e.g., a hook) on its free end (as shown in FIG. 6). The movable component 600 can travel in opposing directions 602 and 900, and is size and shaped to fit in and travel through the internal feature 412 of the implant 400. The gripper or other connector is provided for coupling the tool 500 to a free end 604 of the insert mask 504 and facilitate the pulling of the insert mask 504 in direction 900. Since the opposing end 606 is secured to a structure 604, this pulling by the tool 500 causes the insert mask 504 to stretch as shown in FIGS. 9-11. When the insert mask 504 is released from the gripper or other connector of the tool 500 and/or from the structure 604, it expands as shown in FIG. 12 so that it is securely retained inside the internal feature 412 and provides a seal so that fluids and/or particles are not able to enter the internal feature 412. The present solution is not limited to the particulars of FIGS. 5-12. Other tools and/or insert mask designs can be employed in accordance with a given application.

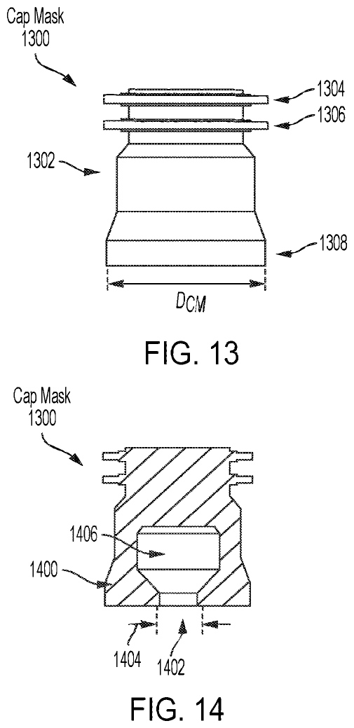

Referring again to FIG. 3, method 300 continues with 308 where a mask is loaded on the implant to provide a cap-implant assembly. Illustrations of a cap mask 1300 are provided in FIGS. 13-14. Cap mask 1300 is formed of a resilient material that can deform from an initial state and return to the initial state. The resilient material can include, but is not limited to, rubber. The deformation can include, but is not limited to, expansion, stretching, bending, and/or compression. Cap mask 1300 comprises a body 1302 with an aperture 1400 formed therein. Aperture 1400 extends only partially through the body 1302, and has an insert opening 1402 with a diameter 1404. The size of diameter 1404 is variable since the cap mask 1300 is formed of a resilient material. The importance of the variable diameter 1404 of insert opening 1402 will become evident as the discussion progresses. The internal space 1406 of the aperture 1400 is sized and shaped to receive the head 402 and neck 404 of implant 400. Other features 1304, 1306, 1308 are provided with cap mask 1300 to facilitate coupling and retention of the cap mask 1300 in a holder as will become evident as the discussion progresses.

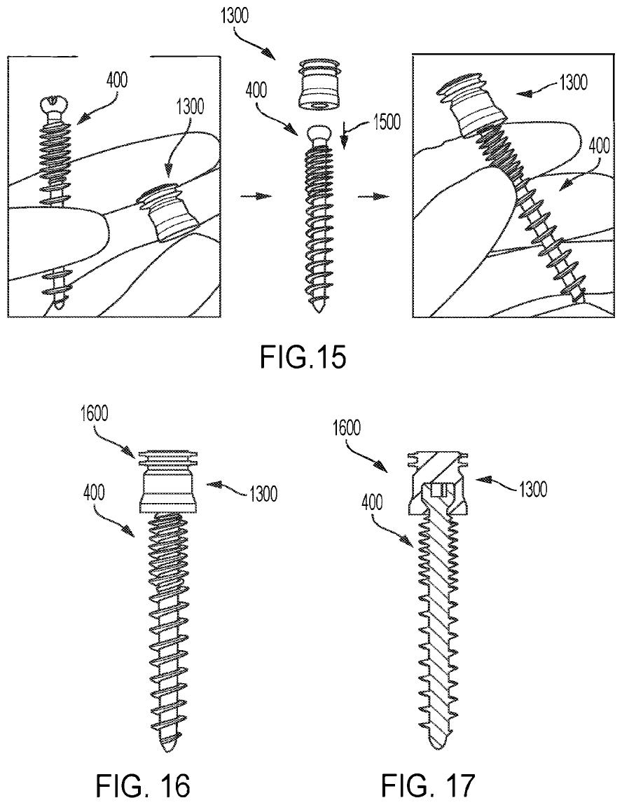

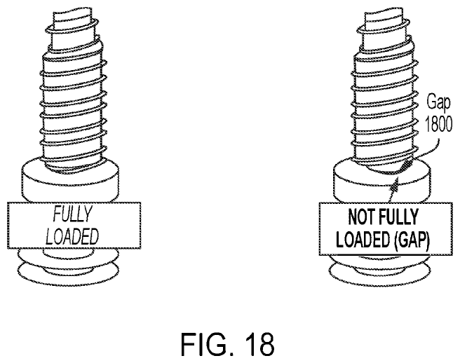

The process of block 308 in which the cap mask 1300 is loaded onto the implant 400 is shown in FIG. 15. The cap mask 1300 is aligned with and pressed against the head 402 of the implant 400. As a consequence of the cap mask 1300 being pressed in direction 1500, the head 402 of the implant 400 causes the diameter 1404 of the insert opening 1402 to increase whereby the head 402 is able to slide through the insert opening 1402 and into the internal space 1406 of the aperture 1400. The diameter 1404 of the insert opening 1402 decreases when the head 402 fully resides in the internal space 1406 of the aperture 1400. The decrease of diameter 1404 causes the cap mask 1300 to tightly encompass and fit around the neck 404 of the implant so as to form a seal through which fluids and/or particles are unable to pass. The combination of the cap mask and the holder forms the seal through which fluids are unable to pass. Fluids may still pass when the cap mask is used alone. Illustrations are provided in FIGS. 16-17 showing the cap mask 1300 loaded onto the implant 400.

Referring again to FIG. 3, method 300 continues with 310 where a check is performed to ensure that the cap mask is fully loaded on the implant. Illustrations are provided in FIG. 18 showing the cap mask in a fully loaded state and a non-fully loaded state. In the non-fully loaded state, a gap 1800 is provided between the implant 400 and the internal surface of the cap mask's insert opening 1402. The seal is not provided between the cap mask 1300 and the implant in this non-fully loaded state. In the event that the cap mask is not fully loaded onto the implant, the operations of 308 may be performed once again.

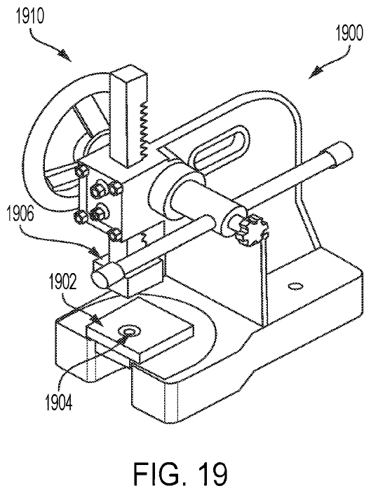

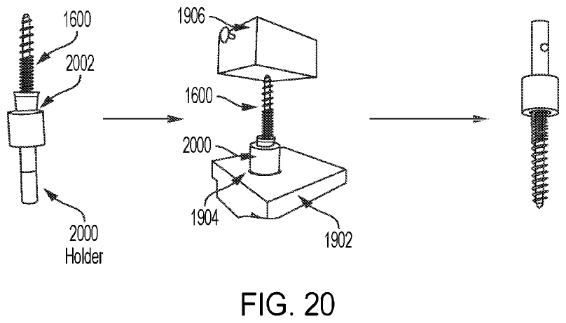

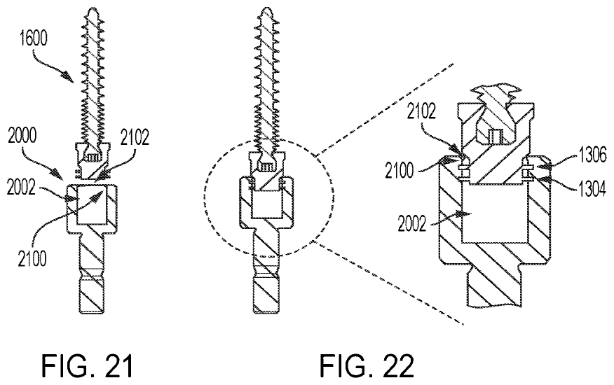

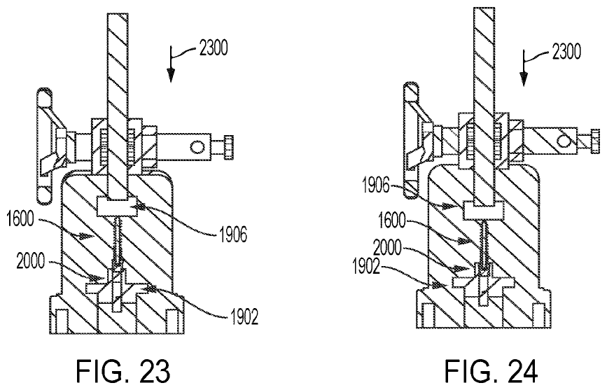

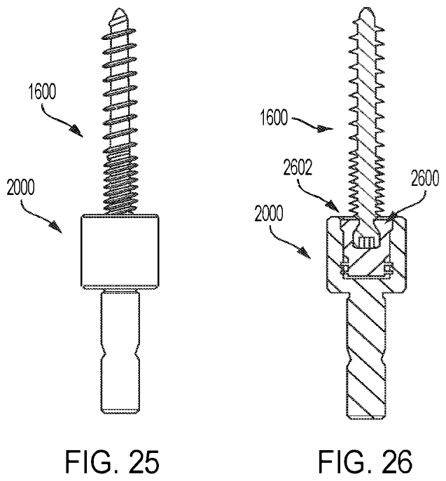

Next in 312, the cap-implant assembly 1600 is loaded into a holder, for example, using a tool 1900 shown in FIG. 19. The manner in which this loading is achieved is shown in FIG. 20. FIGS. 21-27 will also be referenced in this discussion of block 312. First, as shown in FIG. 20, the cap-implant assembly 1600 is aligned with a holder 2000 and pushed into an aperture 2002 of the holder 2000. The pushing force on the cap-implant assembly 1600 may be discontinued when flanges 1304, 1306 of the cap mask 1300 slide past a lip or flange 2100 formed at the opening 2102 of aperture 2002. An illustration of the flanges 1304, 1306 inserted into aperture 2002 past lip/flange 2100 is provided in FIG. 22. When this occurs, the holder (with the partially inserted cap-implant assembly) is placed in an aperture 1904 of the tool's holder support structure 1902. An actuator 1910 is actuated (for example, rotated in the clockwise direction) to cause the tool's engagement member 1906 to move in direction 2300 towards the cap-implant assembly and come in contact with the tip 414 of the implant 400 as shown in FIG. 23. The engagement member 1906 may comprise an indent for receiving and/or at least partially encompassing the implant's tip such that the tip is not damaged or otherwise deformed when the tool is further actuated. The actuator 1910 is further actuated as shown in FIG. 24 so that the engagement member 1906 applies a pushing force against the cap-implant assembly 1600 in direction 2300 toward holder 2000. This pushing force is discontinued when the cap-implant assembly is fully loaded into the holder. In the fully loaded state, the cap mask 1300 is fully inserted into aperture 2002 of the holder 2000 such that a surface 2600 of the cap mask resides below the flange, lip and/or surface 2602 of the holder, as shown in FIGS. 25-26.

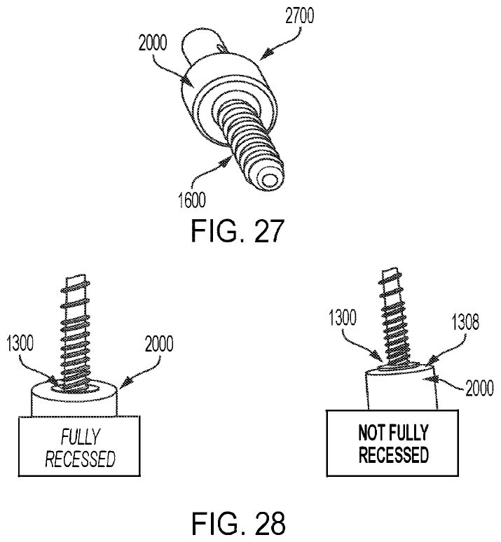

Referring again to FIG. 3, method 300 continues with 314 where a check is performed that the cap-implant assembly is fully loaded into the holder. Illustrations are provided in FIG. 28 showing the cap-implant assembly 1600 in a fully loaded state (shown in left illustration) and a non-fully loaded state (shown in right illustration). In the non-fully loaded state, a feature 1308 of the cap mask extends out and away from the holder 2000. This is undesirable because consistent compression on the cap mask 1600 during surface roughening process(es) is not necessarily maintained. As can be seen in FIG. 13, feature 1308 of the cap mask 1300 has a larger diameter Dcm than the diameter of other portions of the cap mask body 1302. This enlarged diameter is provided to create the seal when the cap-implant assembly 1600 is fully loaded into the holder 2000. The operations of block 312 may be performed again in the event that the cap-implant assembly is not fully loaded into the holder.

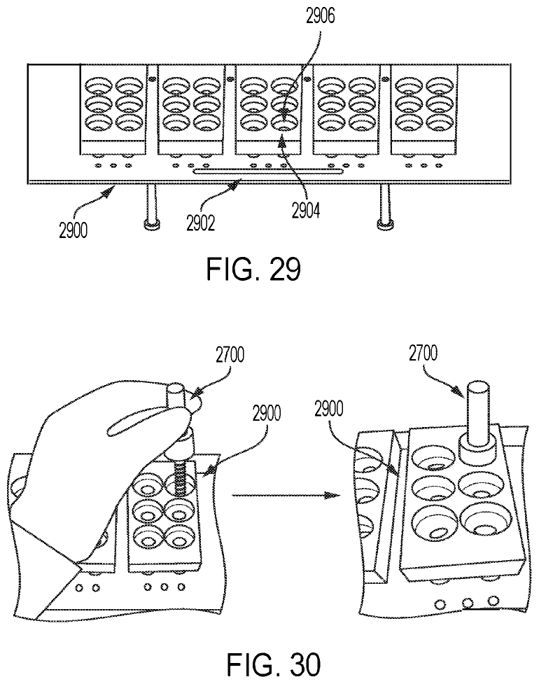

Upon completing the check of block 314, method 300 continues with 316 where the cap-implant-holder assembly 2700 is loaded into a tray. An illustrative tray 2900 is shown in FIG. 29. The tray 2900 comprises a support structure 2902 with apertures 2904 formed therein that are sized and shaped to receive the shank 416 of the implant 400. The shank 416 of the implant 400 is inserted into and through an aperture 2904 of the tray 2900 until the holder 2000 comes in contact with and rests on a ledge 2906 of the tray 2900, as shown in FIG. 30. The tray 2900 structurally supports the cap-implant-holder assembly 2700 in an upright position shown in FIG. 30.

Next in 318, a surface roughing process is performed to create micron scale valleys on an exposed surface of the implant. The surface rouging process can include, but is not limited to, grit blasting and/or chemical etching. Illustrative surface roughing processes that can be used here are described in U.S. Pat. No. 8,814,939 to Ulrich, Jr. et al. and U.S. Patent Publication No. 2012/0316650 to Ullrich, Jr. et al., the entire contents of which are incorporated by reference. The cap-implant-holder assembly 2700 is removed from the tray 2900 when the grit blasting process is completed as shown by block 320 of FIG. 3.

The cap-implant-holder assembly 2700 is then placed in an etching fixture as shown by block 322 of FIG. 3. Another surface roughing process is performed in block 324 to produce nanoscale striations within the micron scale values. This surface roughing process can include, but is not limited to, acid etching, alkaline etching, and/or other chemical etching process. Illustrative surface roughing processes that can be used here are described in above-mentioned U.S. Pat. No. 8,814,939 to Ulrich, Jr et al. and U.S. Patent Publication No. 2012/0316650 to Ullrich, Jr. et al. The cap-implant-holder assembly 2700 can be partially or fully submerged in fluid during the surface roughing process of 324. The cap-implant-holder assembly is rinsed upon completion of the surface rouging process, as shown by block 326 of FIG. 3. Thereafter, the cap-implant-holder assembly is removed from the etching fixture in 328.

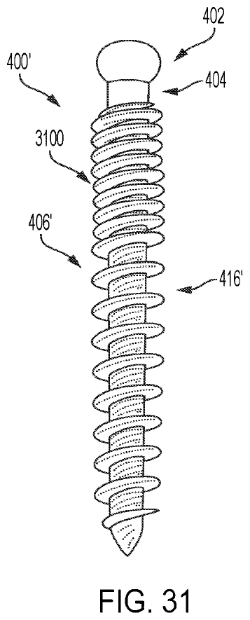

In 330, the cap mask 1300 and holder 2000 are removed from the implant. The implant is then rinsed and dried in 332. The implant now has a roughened surface on its shank and threads to stimulate osseointegration. An illustration of an implant 400′ with the roughened surface 3100 on its shank 416′ and threads 406′ is shown in FIG. 31. The head 402 and neck 404 still have their anodized surface treatment due to its protection by the cap mask during the surface roughing process of block 318 and the surface roughing process of block 324. No material loss occurred in the flat width, head profile and neck diameter.

Subsequently, block 334 is performed where method 300 ends or other operations are performed. These other operations can involve: returning to 302; and/or surgically placing the implant 400′ in a person's body. Any known or to be known technique for surgically placing an implant in a person's body can be used here.

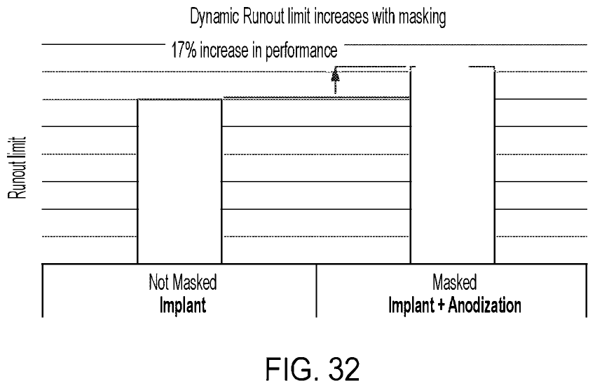

Referring to FIG. 32, there is provided a graph showing results from mechanical testing. The graph demonstrates the utility of masking for increasing a runout limit of the implant.

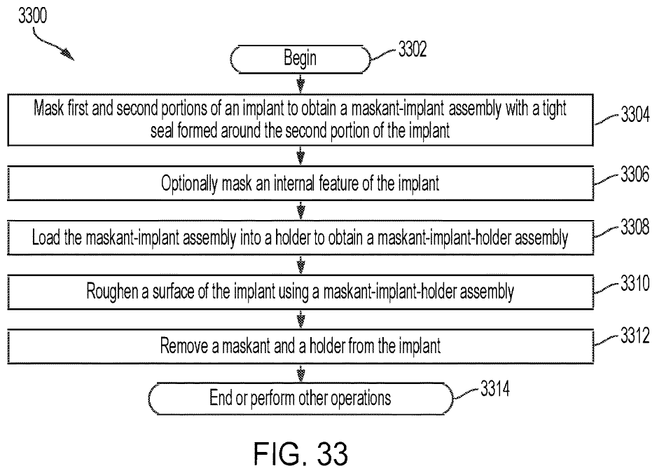

FIG. 33 provides a flow diagram of another illustrative method 3300 for adding surface roughness to an implant (e.g., implant 100 of FIGS. 1 and/or 400 of FIG. 4). Method 3300 begins with 3302 and continues with 3304 where first and second portions of the implant are masked to obtain a maskant-implant assembly with a tight seal formed around the second portion of the implant. The implant may comprise a screw. The first portion may comprise a head of the screw. The second portion may comprise a neck of the screw, and the third portion may comprise a shaft and threads of the screw. The tight seal may comprise a vapor and/or water tight seal.

In some scenarios, the masking of block 3304 involves loading a cap mask on the first and second portions of the implant by: causing a cap mask to at least partially deform from an initial state so that the first portion of the implant is able to slide through an insert opening and into an internal space of the cap mask; and allowing the cap mask to at least partially return to the initial state whereby the first portion of the implant resides in the internal space of the cap mask and the cap mask encompasses and tightly fits around the second portion of the implant.

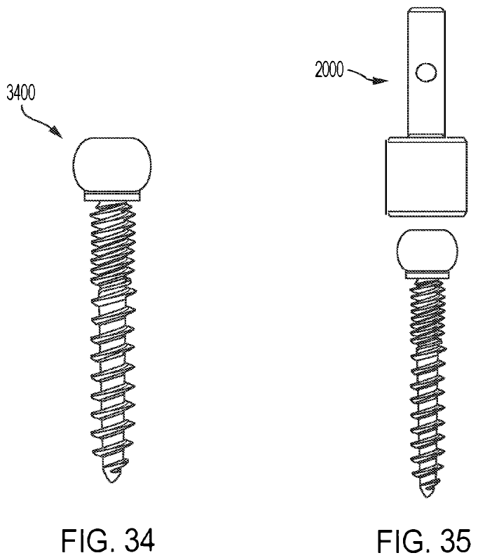

In those and/or other scenarios, the masking of block 3304 involves dipping the first and second portions of the implant into a liquid maskant at a specified depth; removing the first and second portions of the implant from the liquid maskant; and allowing the liquid maskant to cure. The liquid maskant may be cured using an external energy source (e.g., UV light source). As a result of the curing, a semi-flexible mask 3400 is provided around the first and second portions of the implant. Illustrative semi-flexible mask 3400 provided on an implant is shown in FIG. 34.

In those or other scenarios, the masking of block 3304 involves applying a putty maskant on the first and second portions of the implant.

Optional block 3306 involves masking an internal feature of the implant using an insert mask. This masking can be achieved by: stretching the insert mask; pulling the insert mask through an aperture formed in the implant; and releasing the insert mask so that the insert mask at least partially unstretches and seals the aperture.

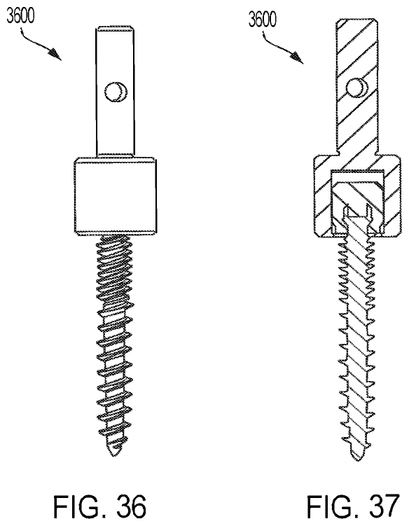

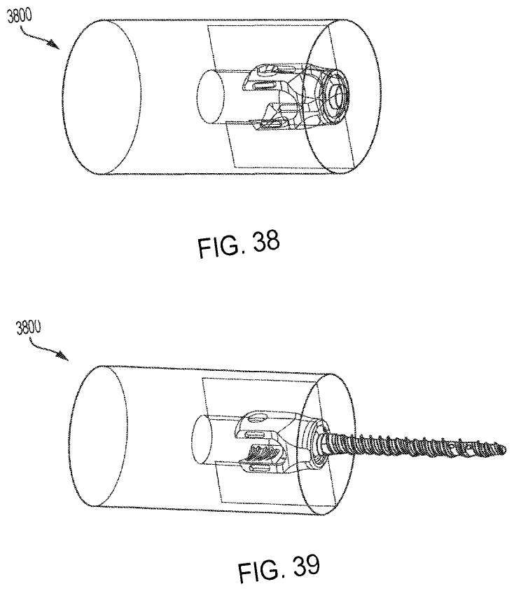

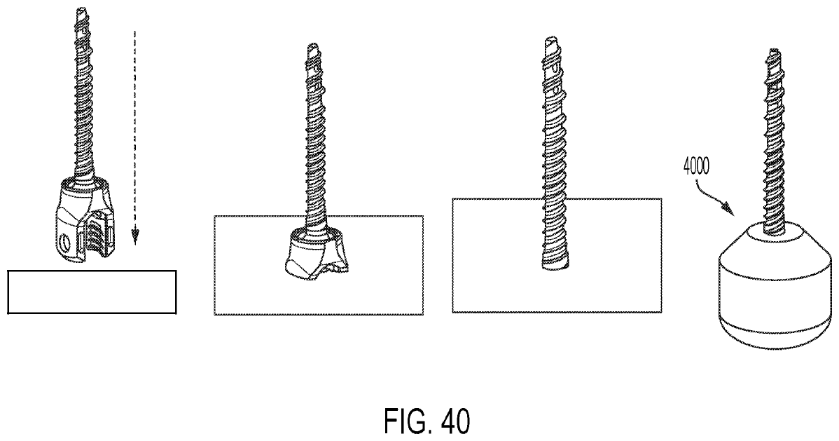

In block 3308, the maskant-implant assembly is loaded into a holder (e.g., holder 2000) to obtain a maskant-implant-holder assembly (e.g., cap-implant-holder assembly 2700 of FIG. 27 or maskant-implant-holder assembly 3600 of FIG. 36).

This loading can be achieved by: applying a first pushing force on the maskant-implant assembly to cause a maskant (e.g., a cap mask) to slide through an opening of an aperture formed in the holder; discontinuing application of the first pushing force when at least one flange of the maskant (e.g., cap mask) slides past a flange formed at the opening of the aperture formed in the holder; using a tool to apply a second pushing force to the maskant-implant assembly to cause the maskant (e.g., cap mask) to further slide into the aperture formed in the holder; and discontinuing application of the second pushing force when the maskant (e.g., cap mask) is fully loaded in the aperture formed in the holder. The maskant (e.g., cap mask) entirely resides in the aperture formed in the holder when the maskant-implant assembly is loaded into the holder. The maskant may be retained in the aperture formed in holder by using the flange formed at the opening of the aperture formed in the holder to provide a hold on the maskant and/or suction created at least partially by the at least one flange of the maskant.

In the cured mask scenarios, the cured mask conforms to the holder internal channel, including slots to retain the maskant-implant in the holder during surface roughening process(es). In the putty mask scenarios, the putty is placed in the holder such that it conforms to the holder opening and fills the slots. The putty is then allowed to cure or be cured using an external energy source (e.g., a UV light source). The cured putty is hardened and secures the implant to the holder.

In block 3310, a surface of the second portion and/or a third portion of implant is roughened using a maskant-implant-holder assembly. The roughening creates microscale features on a surface of the third portion of the implant and/or nanoscale features within or around the microscale features. The geometric dimensions of the first and/or second portions of the implant may be the same before and after the roughening of the surface of the third portion.

In block 3312, the maskant and holder are removed from the implant. The anodized surface finish remains on the first and second portions of the implant and has been removed from the third portion of the implant. The cured maskant may be removed from the implant by submerging at least a portion of the maskant-implant assembly or the maskant-implant-holder assembly in a solution that dissolves the maskant or allows the maskant to be removed from the implant. Additionally or alternatively, the maskant is peeled off of the implant. Subsequently, method 3300 continues to block 3314 where method 3300 ends or other operations are performed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

Example 1. A method for adding surface roughness to an implant with an anodized surface finish, comprising:
a. masking first and second portions of the implant to obtain a maskant-implant assembly with a tight seal formed around the second portion of the implant;
b. roughening a surface of a third portion of implant using a maskant-implant-holder assembly; and
c. removing a maskant and a holder from the implant, wherein the anodized surface finish remains on the first and second portions of the implant and has been removed from the third portion of the implant.

Example 2. The method according to claim 1, wherein the implant comprises a screw, the first portion comprises a head of the screw, the second portion comprises a neck of the screw, and the third portion comprises a shaft and threads of the screw.

Example 3. The method according to any of claims 1 to 2, further comprising masking an internal feature of the implant using an insert mask.

Example 4. The method according to claim 3, wherein the masking the internal feature of the implant using the insert mask comprises:
a. stretching the insert mask;
b. pulling the insert mask through an aperture formed in the implant; and
c. releasing the insert mask so that the insert mask at least partially unstretches and seals the aperture.

Example 5. The method according to any of claims 1-4, wherein the masking the first and second portions of the implant comprises loading a cap mask on the first and second portions of the implant by:
a. causing a cap mask to at least partially deform from an initial state so that the first portion of the implant is able to slide through an insert opening and into an internal space of the cap mask; and
b. allowing the cap mask to at least partially return to the initial state whereby the first portion of the implant resides in the internal space of the cap mask and the cap mask encompasses and tightly fits around the second portion of the implant.

Example 6. The method according to any of claims 1 to 5, further comprising loading the maskant-implant assembly into the holder by:
a. applying a first pushing force on the maskant-implant assembly to cause a cap mask to slide through an opening of an aperture formed in the holder;
b. discontinuing application of the first pushing force when at least one flange of the cap mask slides past a flange formed at the opening of the aperture formed in the holder;
c. using a tool to apply a second pushing force to the maskant-implant assembly to cause the cap mask to further slide into the aperture formed in the holder; and
d. discontinuing application of the second pushing force when the cap mask is fully loaded in the aperture formed in the holder.

Example 7. The method according to claim 6, wherein the cap mask entirely resides in the aperture formed in the holder when the maskant-implant assembly is loaded into the holder.

Example 8. The method according to any of claims 6 to 7, further comprising retaining the cap mask in the aperture formed in holder by using the flange formed at the opening of the aperture formed in the holder to provide a hold on the cap mask and suction created at least partially by the at least one flange of the cap mask.

Example 9. The method according to any of claims 1 to 8, wherein the roughening creates microscale features on a surface of the third portion of the implant.

Example 10. The method according to any of claims 1 to 9, wherein the roughening further creates nanoscale features within or around the microscale features.

Example 11. The method according to any of claims 1 to 10, wherein geometric dimensions of the first and second portions of the implant are the same before and after the roughening of the surface of the third portion.

Example 12. The method according to any of claims 1 to 11, wherein said masking the first and second portions of the implant maintains mechanical integrity of the implant.

Example 13. A system for use with an implant during a surface roughening process, comprising:
a. a cap mask configured to maintain geometric dimensions and an anodized surface finish of first and second portions of the implant during the surface roughening process in which surface roughness is added to a third portion of the implant;
b. a holder with an aperture sized and shaped to receive the cap mask in a manner that facilitates creation of a seal around the second portion of the implant; and
c. a structure provided with the cap mask to facilitate retention of the cap mask in the holder when the cap mask is fully inserted into the aperture of the holder.

Example 14. The system according to claim 13, wherein the cap mask and holder are configured to allow removal of the anodized surface finish from the third portion of the implant during the surface roughening process.

Example 15. An implant, comprising:
a. a first portion with an anodized surface finish;
b. a second portion that is connected to the first portion and has the anodized surface finish; and
c. a third portion that is connected to the second portion, absent of the anodized surface finish, and has a surface roughness comprising microscale features and nanoscale features within or around the microscale features;
d. wherein geometric dimensions of the first and second portions of the implant are the same before, during and after addition of the surface roughness to the third portion of the implant.

1-15. (canceled)

16. A method for adding surface roughness to an implant with an anodized surface finish, comprising:
masking first and second portions of the implant to obtain a maskant-implant assembly with a tight seal formed around the second portion of the implant;
roughening a surface of a third portion of implant using a maskant-implant-holder assembly; and
removing a maskant and a holder from the implant, wherein the anodized surface finish remains on the first and second portions of the implant and has been removed from the third portion of the implant.

17. The method according to claim 16, wherein the implant comprises a screw, the first portion comprises a head of the screw, the second portion comprises a neck of the screw, and the third portion comprises a shaft and threads of the screw.

18. The method according to claim 16, further comprising masking an internal feature of the implant using an insert mask.

19. The method according to claim 18, wherein the masking the internal feature of the implant using the insert mask comprises:
stretching the insert mask;
pulling the insert mask through an aperture formed in the implant; and
releasing the insert mask so that the insert mask at least partially unstretches and seals the aperture.

20. The method according to claim 16, wherein the masking the first and second portions of the implant comprises loading a cap mask on the first and second portions of the implant by:
causing a cap mask to at least partially deform from an initial state so that the first portion of the implant is able to slide through an insert opening and into an internal space of the cap mask; and
allowing the cap mask to at least partially return to the initial state whereby the first portion of the implant resides in the internal space of the cap mask and the cap mask encompasses and tightly fits around the second portion of the implant.

21. The method according to claim 16, further comprising loading the maskant-implant assembly into the holder by:
applying a first pushing force on the maskant-implant assembly to cause a cap mask to slide through an opening of an aperture formed in the holder;
discontinuing application of the first pushing force when at least one flange of the cap mask slides past a flange formed at the opening of the aperture formed in the holder;
using a tool to apply a second pushing force to the maskant-implant assembly to cause the cap mask to further slide into the aperture formed in the holder; and
discontinuing application of the second pushing force when the cap mask is fully loaded in the aperture formed in the holder.

22. The method according to claim 21, wherein the cap mask entirely resides in the aperture formed in the holder when the maskant-implant assembly is loaded into the holder.

23. The method according to claim 21, further comprising retaining the cap mask in the aperture formed in holder by using the flange formed at the opening of the aperture formed in the holder to provide a hold on the cap mask and suction created at least partially by the at least one flange of the cap mask.

24. The method according to claim 16, wherein the roughening creates microscale features on a surface of the third portion of the implant.

25. The method according to claim 24, wherein the roughening further creates nanoscale features within or around the microscale features.

26. The method according to claim 16, wherein geometric dimensions of the first and second portions of the implant are the same before and after the roughening of the surface of the third portion.

27. The method according to claim 16, wherein said masking the first and second portions of the implant maintains mechanical integrity of the implant.

28. A system for use with an implant during a surface roughening process, comprising:
a cap mask configured to maintain geometric dimensions and an anodized surface finish of first and second portions of the implant during the surface roughening process in which surface roughness is added to a third portion of the implant;
a holder with an aperture sized and shaped to receive the cap mask in a manner that facilitates creation of a seal around the second portion of the implant; and
a structure provided with the cap mask to facilitate retention of the cap mask in the holder when the cap mask is fully inserted into the aperture of the holder.

29. The system according to claim 28, wherein the surface roughening process comprises at least one of grit blasting and acid etching.

30. The system according to claim 28, wherein the implant comprises a screw, the first portion comprises a head of the screw, the second portion comprises a neck of the screw, and the third portion comprises a shaft and threads of the screw.

31. The system according to claim 28, further comprising an insert mask configured to mask and seal an internal feature of the implant during the surface roughening process.

32. The system according to claim 31, wherein the insert mask is formed of a resilient material that deforms when a pulling force is applied thereto and undeforms when the pulling force is no longer being applied thereto.

33. The system according to claim 28, wherein the cap mask and holder are configured to allow removal of the anodized surface finish from the third portion of the implant during the surface roughening process.

34. The system according to claim 28, wherein the cap mask is configured to:
at least partially deform from an initial state whereby an insert opening of the cap mask is enlarged to a size and shape for slidingly receiving the first portion of the implant; and
at least partially return to the initial state whereby the insert opening of the cap mask decreases in size to cause the cap mask to encompass and tightly fit around the second portion of the implant.

35. The system according to claim 28, wherein holder comprises:
an aperture sized and shaped to receive the cap mask; and
at least one flange that is formed at an opening of the aperture and configured to facilitate retention of the cap mask when inserted into the holder.

36. The system according to claim 35, wherein at least one flange of the holder is additionally configured to provide an indication of when a suction structure of the cap mask has been inserted into the aperture of the holder.

37. The system according to claim 36, wherein the suction structure of the cap mask is configured to create suction to facilitate retention of the cap mask in the aperture formed in the holder.

38. An implant, comprising:
a first portion with an anodized surface finish;
a second portion that is connected to the first portion and has the anodized surface finish; and
a third portion that is connected to the second portion, absent of the anodized surface finish, and has a surface roughness comprising microscale features and nanoscale features within or around the microscale features;
wherein geometric dimensions of the first and second portions of the implant are the same before, during and after addition of the surface roughness to the third portion of the implant.
